(19) United States
(12) Patent Application Publication
(10) Pub. No.: US 2019/0120829 A1
(43) Pub. Date: Apr. 25, 2019

(54) MIXING METHOD AND MIXING APPARATUS FOR PARTICLE AGGLUTINATION

(71) Applicant: DELTA ELECTRONICS, INC., Guishan Dist. (TW)

(72) Inventors: Chi-Han CHIOU, Taoyuan City (TW); Kuan-Yu CHEN, Taoyuan City (TW); Ching-Yu CHANG, Taoyuan City (TW)

(21) Appl. No.: 15/895,842

(22) Filed: Feb. 13, 2018

(30) Foreign Application Priority Data
Oct. 25, 2017 (TW) 106136750

(51) Int. Cl. G01N 33/53; G01N 15/14; B01L 3/00; G01N 33/543; B01F 13/00; G01N 1/38; G01N 33/80

(52) U.S. Cl. CPC G01N 33/5304; G01N 15/1484; B01L 3/502792; G01N 2015/1081; B01F 13/0064; G01N 1/38; G01N 33/80; G01N 33/54333

(57) ABSTRACT

A mixing method for particle agglutination includes the following steps of: dropping testing materials into an accommodating recess at one end of a channel structure; pressing a flexible layer on the other end of the channel structure; and releasing the flexible layer to an initial position after pressing the flexible layer such that a negative pressure is generated by an air chamber that is covered by the flexible layer and draws the testing materials that are in the accommodating recess to move toward the air chamber along a diverging channel of the channel structure. The testing materials are mixed with each other in the diverging channel, in which the depth of the diverging channel is gradually increased from the accommodating recess to the air chamber.

### RELATED APPLICATIONS

This application claims priority to Taiwan Application Serial Number 106136750, filed Oct. 25, 2017, which is herein incorporated by reference.

### BACKGROUND

### Field of Invention

The present invention relates to a mixing method and a mixing apparatus for particle agglutination.

### Description of Related Art

In general, there are three blood types testing methods to mix blood and an antibody, including a test-tube immediate centrifugal method, a solid microplate method and a gel column agglutination method. In the test-tube immediate centrifugal method, blood and antibody are mixed in a test tube. If the blood is agglutinative, the blood is gathered to form blood agglutination that precipitates at the bottom of the test tube. However, the test-tube immediate centrifugal method has some problems, such as lack of standardization process of blood typing test and intensive time and labor consumption.

Moreover, in the solid microplate method, blood and an antibody are placed in microplates. Each of the microplates has many edges with a concave portion in the arc hole. The blood and the antibody are able to be mixed for a long time for shaking the microplates. If the blood is agglutinative, the blood is gathered into blood agglutination, and jammed in concave portion. If the blood is not agglutinative, blood cells are gathered in the central portion of the microplate. However, this solid microplate method is only a semi-quantitative test for a rough estimation. In addition, the solid microplate method also suffers the disadvantages of long testing time and the necessary pretreatment of the specimen.

As refer to the gel column agglutination method, a column is filled with gel as a gel column, and the blood and the antibody are placed on the top of the gel. The blood and the antibody are mixed through a centrifugal method. If the blood is agglutinative, the blood is gathered into blood agglutination and jammed in the top or central portion of the gel. If it is not agglutinative, the blood is centrifuged to the bottom of the gel. However, the detection card with the gel columns used in the gel column agglutination method is expensive, and a centrifuge is required in the gel column agglutination method. In addition, the gel column agglutination method also has the deficiencies of long testing time and the necessary pretreatment of the specimen.

### SUMMARY

An aspect of the present invention is to provide a mixing method for particle agglutination with advantages of reducing testing steps and testing time, and improving operation convenience.

According to an embodiment of the present invention, a mixing method for particle agglutination includes the following steps of: (a) dropping testing materials into an accommodating recess at one end of a channel structure; (b) pressing a flexible layer on the other end of the channel structure; and (c) releasing the flexible layer to its initial position after the flexible layer is pressed such that a negative pressure is generated in an air chamber that is covered by the flexible layer to draw the testing materials that are in the accommodating recess to move toward the air chamber along a diverging channel of the channel structure. The testing materials are mixed with each other in the diverging channel, in which a depth of the diverging channel is gradually increasing along a direction from the accommodating recess to the air chamber.

In one embodiment of the present invention, a magnitude of a velocity of pressing the flexible layer is greater than or equal to a magnitude of a velocity of releasing the flexible layer.

In one embodiment of the present invention, a magnitude of a velocity of pressing the flexible layer is smaller than or equal to a magnitude of a velocity of releasing the flexible layer.

In one embodiment of the present invention, step (b) includes programming a pressing device to control a downward velocity of a pressing head of the pressing device; and moving the pressing head downward to press the flexible layer.

In one embodiment of the present invention, a magnitude of the downward velocity is in a range from 0.29 mm/s to 0.83 mm/s or from 2.50 mm/s to 8.33 mm/s.

In one embodiment of the present invention, the mixing method for particle agglutination further includes programming the pressing device to control a position of the pressing head and a frequency of pressing the flexible layer.

In one embodiment of the present invention, step (c) includes programming a pressing device to control an upward velocity of a pressing head of the pressing device; and moving the pressing head upward to release the flexible layer to its initial position.

In one embodiment of the present invention, a magnitude of the upward velocity is in a range from 0.29 mm/s to 0.83 mm/s or from 2.50 mm/s to 8.33 mm/s.

In one embodiment of the present invention, the accommodating recess has an inclined surface, and step (c) includes enabling the testing materials to enter the diverging channel along the inclined surface of the accommodating recess.

An aspect of the present invention is to provide a mixing apparatus for particle agglutination with advantages of reducing testing steps and testing time, and improving operation convenience.

According to an embodiment of the present invention, a mixing apparatus for particle agglutination includes a card, a channel structure, and a flexible layer. The channel structure is embedded in the card and has a diverging channel. Two ends of the channel structure respectively have an accommodating recess and an air chamber. The accommodating recess has a first opening. The air chamber has a second opening. The diverging channel is located between the accommodating recess and the air chamber, and communicates with the accommodating recess and the air chamber. A depth of the diverging channel is gradually increasing along a direction from the accommodating recess to the air chamber. The flexible layer covers the second opening of the air chamber.

In one embodiment of the present invention, the mixing apparatus for particle agglutination further includes a pressing device. The pressing device has a pressing head on the flexible layer. The pressing device is programmed to control a downward velocity of the pressing head when pressing the flexible layer and an upward velocity of the pressing head when releasing the flexible layer to its initial position.

In one embodiment of the present invention, the pressing device includes a motor or a pump connected to the pressing head.

In one embodiment of the present invention, the accommodating recess has an inclined surface, the channel structure has a bottom surface extending to the accommodating recess, and the inclined surface adjoins the bottom surface.

In one embodiment of the present invention, an obtuse angle is formed between the inclined surface of the accommodating recess and the bottom surface of the channel structure.

In one embodiment of the present invention, the diverging channel has a substantially transparent cover.

In the aforementioned embodiments of the present invention, because the channel structure has the diverging channel, the accommodating recess, and the air chamber that communicate with the diverging channel, the flexible layer may be released to its initial position after the flexible layer is pressed, such that a negative pressure is generated in the air chamber to draw the testing materials that are in the accommodating recess to the air chamber. Moreover, a depth of the diverging channel is gradually increasing along a direction from the accommodating recess to the air chamber, and thus the testing materials may be moved toward the air chamber along the diverging channel, and can be ensured to mix with each other in the diverging channel, such that users can conveniently observe whether the agglutination phenomenon of the mixed testing materials occurs in the area of the diverging channel. The fabrication cost of the mixing apparatus for particle agglutination of the present invention is low, and the mixing apparatus can be operated without any centrifugal apparatus. Furthermore, the testing materials can be mixed merely by pressing the flexible layer, thereby reducing testing steps and testing time, and improving operation convenience.

It is to be understood that both the foregoing general description and the following detailed description are by examples, and are intended to provide further explanation of the invention as claimed.

### BRIEF DESCRIPTION OF THE DRAWINGS

The invention can be more fully understood by reading the following detailed description of the embodiments, with reference made to the accompanying drawings as follows:

FIG. 1 is a perspective view of a mixing apparatus for particle agglutination according to one embodiment of the present invention;

FIG. 2 is a cross-sectional view of the mixing apparatus for particle agglutination taken along line 2-2 shown in FIG. 1;

FIG. 3 is a cross-sectional view of testing materials which are dropped in an accommodating recess shown in FIG. 2;

FIG. 4 is a cross-sectional view of a flexible layer shown in FIG. 3 after the flexible layer is pressed;

FIG. 5 is a cross-sectional view of the flexible layer shown in FIG. 4 after the flexible is released to its initial position;

FIG. 6 is a dead volume diagram of the testing materials retained in a channel structure shown in FIG. 5;

FIG. 7 is a top view of a channel structure with testing materials according to one embodiment of the present invention, in which the testing materials form agglutinative particles;

FIG. 8 is a top view of a channel structure with testing materials according to another embodiment of the present invention, in which the testing materials form agglutinative particles;

FIG. 9 is a top view of a channel structure with testing materials according to another embodiment of the present invention, in which the testing materials form agglutinative particles; and

FIG. 10 is a top view of a channel structure with testing materials according to another embodiment of the present invention, in which the testing material form agglutinative particles.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

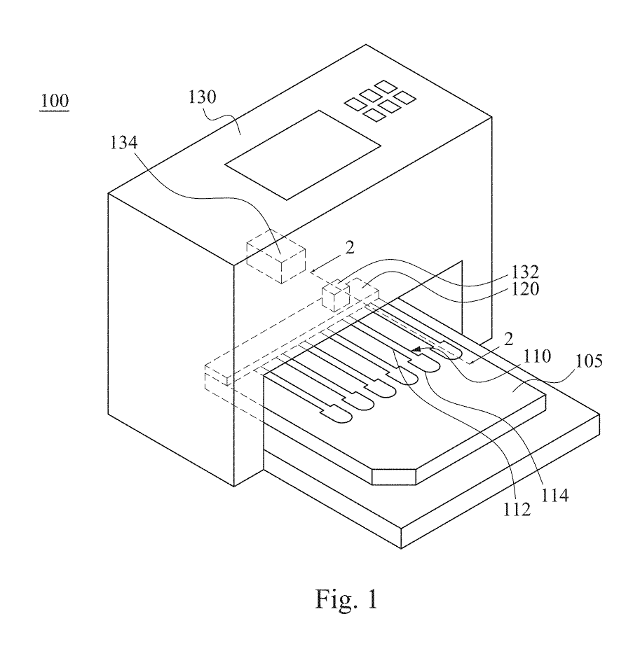

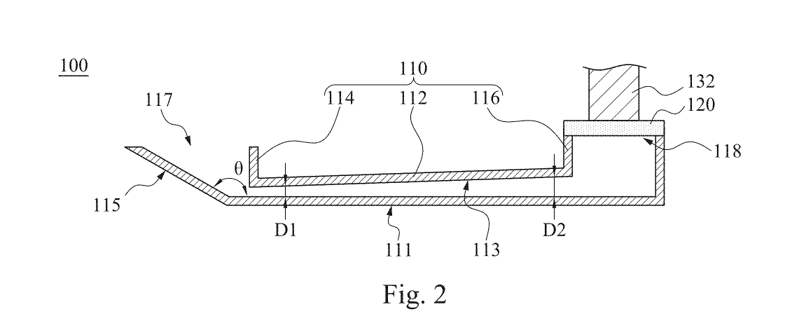

FIG. 1 is a perspective view of a mixing apparatus 100 for particle agglutination according to one embodiment of the present invention. FIG. 2 is a cross-sectional view of the mixing apparatus 100 for particle agglutination taken along line 2-2 shown in FIG. 1. As shown in FIG. 1 and FIG. 2, the mixing apparatus 100 for particle agglutination includes a card 105, a channel structure 110, and a flexible layer 120. The channel structure 110 is embedded in the card 105. The channel structure 110 has a diverging channel 112, and two ends of the channel structure 110 respectively have an accommodating recess 114 and an air chamber 116. The channel structure 110 may be integrally formed as one single piece, but the present invention is not limited to this regard.

The accommodating recess 114 has a first opening 117. The air chamber 116 has a second opening 118. The diverging channel 112 is located between the accommodating recess 114 and the air chamber 116, and communicates with the accommodating recess 114 and the air chamber 116. Furthermore, a depth of the diverging channel 112 is gradually increasing along a direction from the accommodating recess 114 to the air chamber 116. For example, the accommodating recess 114 adjacent to the diverging channel 112 has a depth D1, the accommodating recess 114 adjacent to the air chamber 116 has a depth D2, and the depth D2 is greater than the depth D1. The flexible layer 120 covers the second opening 118 of the air chamber 116, and may be made of a material including foam or rubber.

In this embodiment, the accommodating recess 114 has an inclined surface 115. The channel structure 110 has a bottom surface 111 that extends to the accommodating recess 114, and the inclined surface 115 of the accommodating recess 114 adjoins the bottom surface 111 of the channel structure 110. An obtuse angle θ is formed between the inclined surface 115 of the accommodating recess 114 and the bottom surface 111 of the channel structure 110. The diverging channel 112 has a cover 113, and the cover 113 is substantially transparent, such that users can conveniently observe an agglutination phenomenon in the diverging channel 112.

The channel structure 110 further includes a pressing device 130. The pressing device 130 has a pressing head 132, and has a motor or a pump 134 connected to the pressing head 132. The pressing head 132 is located on the flexible layer 120. The pressing device 130 is programmed to control a downward velocity of the pressing head 132 when pressing the flexible layer 120, and an upward velocity of the pressing head 132 when the flexible layer 120 is released (i.e., recovered) to an initial position. Although the flexible layer 120 shown in FIG. 2 to FIG. 5 is pressed by the pressing head 132 of the pressing device 130, users may directly use their fingers to press the flexible layer 120 without needing to use the pressing device 130, in other embodiments.

The mixing apparatus 100 for particle agglutination may be applied to distinguish ABO blood types and Rh blood types, and may be applied in irregular antibody screening, but the present invention is not limited to this regard. In the following description, a mixing method for particle agglutination by using the mixing apparatus 100 for particle agglutination will be described. In addition, the connection relationships of the elements described above will not be described again hereinafter.

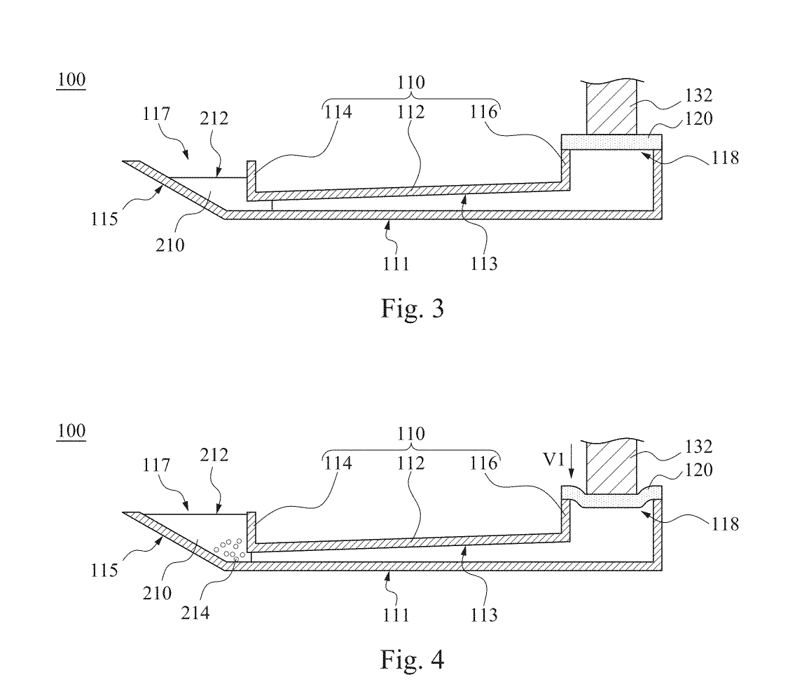

FIG. 3 is a cross-sectional view of testing materials 210 which are dropped in the accommodating recess 114 shown in FIG. 2. FIG. 4 is a cross-sectional view of the flexible layer 120 shown in FIG. 3 after flexible layer 120 is pressed. As shown in FIG. 3 and FIG. 4, the testing materials 210 include a to-be tested material and a discrimination material. The number of to-be tested materials, the number of discrimination materials, and types of the to-be tested material and the discrimination material may be determined as deemed necessary by users. For example, the to-be tested material may include a biological specimen, food, an environmental substance, a microorganism, or combinations thereof, and the discrimination material may include an antibody, an antigen, an indicator, a dye, a biomarker, or combinations thereof.

In operation, the testing materials 210 may be dropped into the first opening 117 of the accommodating recess 114 at one end of the channel structure 110. Thereafter, the flexible layer 120 on the other end of the channel structure 110 is pressed, such that the air in the air chamber 116 is compressed to generate a positive pressure. As a result, the testing materials 210 may move toward the first opening 117 of the accommodating recess 114, and thus a liquid level 212 of the testing materials 210 rises a little, as illustrated in FIG. 4. In this step, the testing materials 210 may have been mixed to form agglutinative particles 214 in the accommodating recess 114. The number and the size of the agglutinative particles 214 of FIG. 4 are merely shown for illustration, and the present invention is not limited to this regard.

In this embodiment, the pressing device 130 (see FIG. 1) may be programmed to control a position and a velocity of the pressing head 132, and a frequency of pressing the flexible layer 120 by the pressing head 132. For example, the programmed pressing device 130 may control a downward velocity V1 of the pressing head 132 to press the flexible layer 120. A magnitude of the velocity V1 may be in a range from 1 mm/s to 10 mm/s, such as in an exemplary range from 2.50 mm/s to 8.33 mm/s. Alternatively, a magnitude of the velocity V1 may be in a range from 0.1 mm/s to 1 mm/s, such as in an exemplary range from 0.29 mm/s to 0.83 mm/s, but the present invention is not limited to this regard.

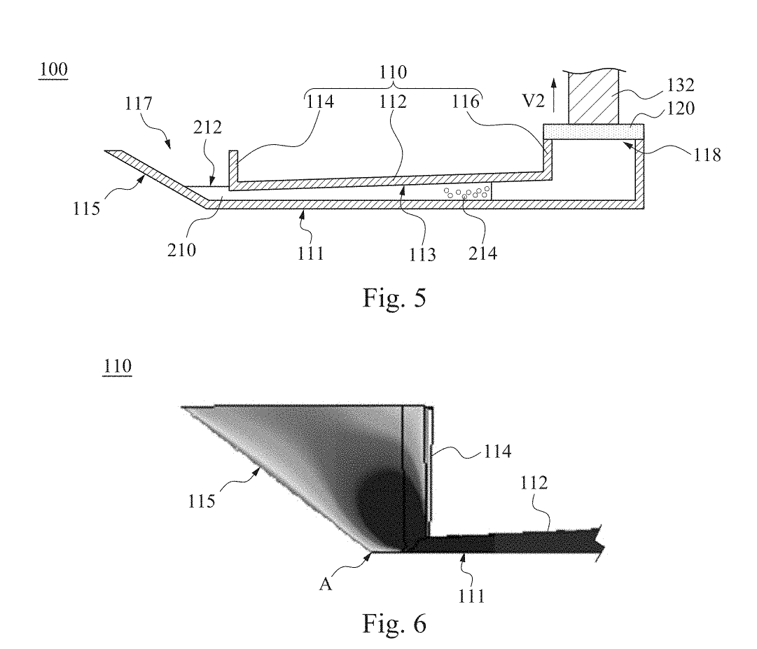

FIG. 5 is a cross-sectional view of the flexible layer 120 shown in FIG. 4 after the flexible layer 120 is released to its initial position. As shown in FIG. 4 and FIG. 5, after the flexible layer 120 is pressed, the flexible layer 120 is released to its initial position, such that a negative pressure is generated in the air chamber 116 that is covered by the flexible layer 120 to further draw the testing materials 210 that are in the accommodating recess 114 to move toward the air chamber 116 along the diverging channel 112 of the channel structure 110. As a result, the testing materials 210 are mixed with each other in the diverging channel 112. In this step, the testing materials 210 have been mixed to form the agglutinative particles 214 in the diverging channel 112. The number and the size of the agglutinative particles 214 of FIG. 5 are merely shown for illustration, and the present invention is not limited to this regard.

A depth of the diverging channel 112 is gradually increasing along a direction from the accommodating recess 114 to the air chamber 116. Therefore, the diverging channel 112 located closer to the air chamber 116 may accommodate more testing materials 210, thereby ensuring that the testing materials 210 are mixed in the diverging channel 112 and do not enter the air chamber 116. Users are able to conveniently observe whether the agglutination phenomenon of the mixed testing materials 210 occurs in the area of the diverging channel 112. The fabrication cost of the mixing apparatus 100 for particle agglutination of the present invention is low, and the mixing apparatus 100 can operate without any centrifugal apparatus. Furthermore, the testing materials 210 may be mixed merely by pressing the flexible layer 120, thereby reducing testing steps and testing time, and improving operation convenience.

In this embodiment, the pressing device 130 may be programmed to control an upward velocity V2 of the pressing head 132 to release the flexible layer 120 to the initial position. A magnitude of the velocity V2 may be in a range from 1 mm/s to 10 mm/s, such as in an exemplary range from 2.50 mm/s to 8.33 mm/s. Alternatively, a magnitude of the velocity V2 may be in a range from 0.1 mm/s to 1 mm/s, such as in an exemplary range from 0.29 mm/s to 0.83 mm/s, but the present invention is not limited to this regard.

FIG. 6 is a dead volume diagram of the testing materials 210 retained in the channel structure 110 shown in FIG. 5. As shown in FIG. 5 and FIG. 6, since the accommodating recess 114 has the inclined surface 115, the testing materials 210 may enter the diverging channel 112 along the inclined surface 115 of the accommodating recess 114 when a negative pressure draws the testing materials 210 into the diverging channel 112, such that the testing materials 210 in the accommodating recess 114 of the channel structure 110 have a small dead volume. Therefore, the testing materials 210 will not silt up an area A adjoining the connection position of the bottom surface 111 and the inclined surface 115, and the proportion of the testing materials 210 entering the diverging channel 112 may be increased, such that the testing materials 210 may be more uniformly mixed, and the accuracy of reading the mixed result can be further improved when users observe above the diverging channel 112.

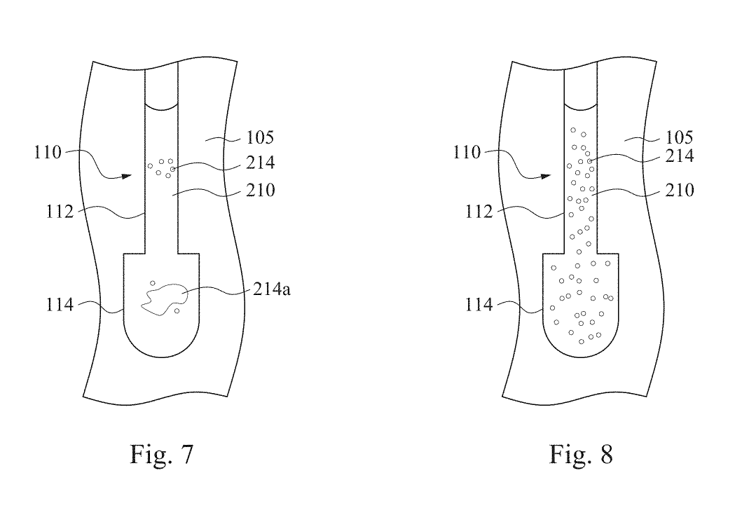

FIG. 7 is a top view of the channel structure 110 with the testing materials 210 according to one embodiment of the present invention, in which the testing materials 210 form the agglutinative particles 214 and 214a. The agglutination phenomenon of FIG. 7 is a result of pressing the flexible layer 120 (see FIG. 4) with a high velocity and releasing the flexible layer 120 (see FIG. 5) to an initial position with a low velocity. A magnitude of the velocity V1 of pressing the 120 (see FIG. 4) is in a range from 1 mm/s to 10 mm/s, such as in an exemplary range from 2.50 mm/s to 8.33 mm/s. A magnitude of the velocity V2 of releasing the flexible layer 120 (see FIG. 5) to the initial position is in a range from 0.1 mm/s to 1 mm/s, such as in an exemplary range from 0.29 mm/s to 0.83 mm/s. In other words, the magnitude of the velocity V1 of pressing the 120 may be greater than the magnitude of the velocity V2 of releasing the flexible layer 120. The flexible layer 120 may be pressed and released by the pressing head 132 of the programmed pressing device 130 (see FIG. 1). As shown in FIG. 7, the agglutinative particle 214a with a larger particle size is located in the accommodating recess 114.

FIG. 8 is a top view of the channel structure 110 with the testing materials 210 according to another embodiment of the present invention, in which the testing materials 210 form agglutinative particles 214. The agglutination phenomenon of FIG. 8 is a result of pressing the flexible layer 120 (see FIG. 4) with a high velocity and releasing the flexible layer 120 (see FIG. 5) to an initial position with a high velocity. A magnitude of the velocity V1 of pressing the 120 (see FIG. 4) is in a range from 1 mm/s to 10 mm/s, such as in an exemplary range from 2.50 mm/s to 8.33 mm/s. A magnitude of the velocity V2 of releasing the flexible layer 120 (see FIG. 5) to the initial position is in a range from 1 mm/s to 10 mm/s, such as in an exemplary range from 2.50 mm/s to 8.33 mm/s. In other words, the magnitude of the velocity V1 of pressing the 120 may be substantially equal to the magnitude of the velocity V2 of releasing the flexible layer 120. The flexible layer 120 may be pressed and released by the pressing head 132 of the programmed pressing device 130 (see FIG. 1). As shown in FIG. 8, the agglutinative particles 214 uniformly distribute in the diverging channel 112 and the accommodating recess 114.

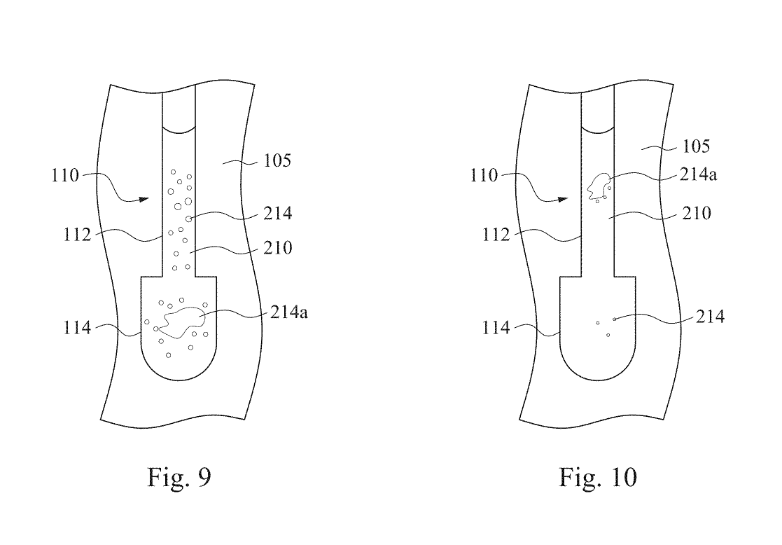

FIG. 9 is a top view of the channel structure 110 with the testing materials 210 according to another embodiment of the present invention, in which the testing materials 210 form the agglutinative particles 214 and 214a. The agglutination phenomenon of FIG. 9 is a result of pressing the flexible layer 120 (see FIG. 4) with a low velocity and releasing the flexible layer 120 (see FIG. 5) to an initial position with a low velocity. A magnitude of the velocity V1 of pressing the 120 (see FIG. 4) is in a range from 0.1 mm/s to 1 mm/s, such as in an exemplary range from 0.29 mm/s to 0.83 mm/s. A magnitude of the velocity V2 of releasing the flexible layer 120 (see FIG. 5) to the initial position is in a range from 0.1 mm/s to 1 mm/s, such as in an exemplary range from 0.29 mm/s to 0.83 mm/s. In other words, the magnitude of the velocity V1 of pressing the 120 may be substantially equal to magnitude of the velocity V2 of releasing the flexible layer 120. The flexible layer 120 may be pressed and released by the pressing head 132 of the programmed pressing device 130 (see FIG. 1). As shown in FIG. 9, the agglutinative particle 214a with a larger particle size is located in the accommodating recess 114.

FIG. 10 is a top view of the channel structure 110 with the testing materials 210 according to another embodiment of the present invention, in which the testing materials 210 form the agglutinative particles 214 and 214a. The agglutination phenomenon of FIG. 10 is a result of pressing the flexible layer 120 (see FIG. 4) with a low velocity and releasing the flexible layer 120 (see FIG. 5) to an initial position with a high velocity. A magnitude of the velocity V1 of pressing the 120 (see FIG. 4) is in a range from 0.1 mm/s to 1 mm/s, such as in an exemplary range from 0.29 mm/s to 0.83 mm/s. A magnitude of the velocity V2 of releasing the flexible layer 120 (see FIG. 5) to the initial position is in a range from 1 mm/s to 10 mm/s, such as in an exemplary range from 2.50 mm/s to 8.33 mm/s. In other words, the magnitude of the velocity V1 of pressing the 120 may be smaller than the magnitude of the velocity V2 of releasing the flexible layer 120. The flexible layer 120 may be pressed and released by the pressing head 132 of the programmed pressing device 130 (see FIG. 1). As shown in FIG. 10, the agglutinative particle 214a with a larger particle size is located in the diverging channel 112.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention covers modifications and variations of this invention provided they fall within the scope of the following claims.

What is claimed is:

1. A mixing method for particle agglutination, comprising steps of:
(a) dropping a plurality of testing materials into an accommodating recess at one end of a channel structure;
(b) pressing a flexible layer at the other end of the channel structure; and
(c) releasing the flexible layer to its initial position after the flexible layer is pressed, such that a negative pressure is generated in an air chamber that is covered by the flexible layer to draw the testing materials that are in the accommodating recess to move toward the air chamber along a diverging channel of the channel structure and the testing materials are mixed with each other in the diverging channel, wherein a depth of the diverging channel is gradually increasing along a direction from the accommodating recess to the air chamber.

2. The mixing method for particle agglutination of claim 1, wherein a magnitude of a velocity of pressing the flexible layer is greater than or equal to a magnitude of a velocity of releasing the flexible layer.

3. The mixing method for particle agglutination of claim 1, wherein a magnitude of a velocity of pressing the flexible layer is smaller than or equal to, a magnitude of a velocity of releasing the flexible layer.

4. The mixing method for particle agglutination of claim 1, wherein the step (b) comprises:
programming a pressing device to control a downward velocity of a pressing head of the pressing device; and
moving the pressing head downward to press the flexible layer.

5. The mixing method for particle agglutination of claim 4, wherein a magnitude of the downward velocity is in a range from 0.29 mm/s to 0.83 mm/s or from 2.50 mm/s to 8.33 mm/s.

6. The mixing method for particle agglutination of claim 4, further comprising:
programming the pressing device to control a position of the pressing head and a frequency of pressing the flexible layer.

7. The mixing method for particle agglutination of claim 1, wherein the step (c) comprises:
programming a pressing device to control an upward velocity of a pressing head of the pressing device; and
moving the pressing head upward to release the flexible layer to the initial position.

8. The mixing method for particle agglutination of claim 7, wherein a magnitude of the upward velocity is in a range from 0.29 mm/s to 0.83 mm/s or from 2.50 mm/s to 8.33 mm/s.

9. The mixing method for particle agglutination of claim 1, wherein the accommodating recess has an inclined surface, and the step (c) comprises:
enabling the testing materials to enter the diverging channel along the inclined surface of the accommodating recess.

10. A mixing apparatus for particle agglutination, comprising:
a card;
a channel structure embedded in the card and having a diverging channel, wherein two ends of the channel structure respectively have an accommodating recess and an air chamber, the accommodating recess has a first opening, the air chamber has a second opening, the diverging channel is located between the accommodating recess and the air chamber and communicates with the accommodating recess and the air chamber, and a depth of the diverging channel is gradually increased along a direction from the accommodating recess to the air chamber; and
a flexible layer covering the second opening of the air chamber.

11. The mixing apparatus for particle agglutination of claim 10, further comprising:
a pressing device having a pressing head on the flexible layer, wherein the pressing device is programmed to control a downward velocity of the pressing head when pressing the flexible layer and an upward velocity of the pressing head when releasing the flexible layer to an initial position.

12. The mixing apparatus for particle agglutination of claim 11, wherein the pressing device comprises a motor or a pump, which is connected to the pressing head.

13. The mixing apparatus for particle agglutination of claim 10, wherein the accommodating recess has an inclined surface, the channel structure has a bottom surface extending to the accommodating recess, and the inclined surface adjoins the bottom surface.

14. The mixing apparatus for particle agglutination of claim 13, wherein an obtuse angle is included between the inclined surface of the accommodating recess and the bottom surface of the channel structure.

15. The mixing apparatus for particle agglutination of claim 10, wherein the diverging channel has a substantially transparent cover.
